(19) United States
(12) Patent Application Publication
(10) Pub. No.: US 2006/0172013 A1
(43) Pub. Date: Aug. 3, 2006

(54) Process for preparing copper oxide-coated antibacterial material

(76) Inventor: Akira Hirai, Kawaguchi-shi (JP)

Correspondence Address: MARGER JOHNSON & MCCOLLOM, P.C., 210 SW MORRISON STREET, SUITE 400, PORTLAND, OR, 97204, US

(21) Appl. No.: 11/129,175

(22) Filed: May 13, 2005

(30) Foreign Application Priority Data
Feb. 3, 2005 (KR) 2005-0010027

(51) Int. Cl. A61K 33/34

(52) U.S. Cl. 424/630; 424/632

(57) ABSTRACT

Disclosed herein is an antibacterial material exhibiting constant, stable, and potent antibacterial, bactericidal and antifungal effects by formation of uniform, hard and thin film-like copper oxide coatings on surfaces of inoxidizable natural materials including mineral materials such as ceramics, glass, stone, tile, pumice and sand, to which a plating method is not easily applicable, or of ceramic products and titanium products, and a process for preparing a copper oxide-coated antibacterial material, comprising dissolving less than or equal to 10% by weight of inorganic copper salt powder or copper sulfate hydrate powder in a polar solvent, in relation to the weight of the polar solvent, so as to prepare a copper ion mixed solution, dipping a target material in the mixed solution, and sintering the target material in which copper ions in the mixed solution were deposited onto the surface of the target material at a predetermined temperature under atmospheric pressure to form a copper oxide coating by change of a copper oxide ionic film into a copper oxide coating on the surface of the material.

The present invention claims the benefit of Korean Patent Application No. 2005-0010027 filed on Feb. 3, 2005, which is hereby incorporated by reference.

### BACKGROUND OF THE INVENTION

1. Field of the Invention

The present invention relates to a process for preparing an antibacterial material for use in various cooking utensils, medical instruments, bathrooms or bathing environments and facilities, which require antibacterial, bactericidal and antifungal properties for sanitation purposes, or for use in various materials to maintain a hygienic environment in vast areas, such as food manufacturing apparatuses and components thereof, animal breeding facilities, etc.

Specifically, the present invention relates to a process for preparing an antibacterial material obtained by forming a uniform, hard and thin film-like copper oxide coating on surfaces of various materials or products, including ceramic materials such as ceramics and potteries, and thermally resistant sand materials, various knife blades and dinner wares made of titanium/ceramics, shape-processed ceramic products such as tiles, and materials such as sand or stones, used in drinking water purification tanks or bathtubs.

2. Description of the Related Art

Conventionally, techniques for producing inorganic antibacterial agents such as copper based agents or the like have been widely used in antibacterial and antifungal fields, and effectiveness of such antibacterial agents has been commonly and broadly acknowledged in the art. As examples of antibacterial techniques utilizing copper, various patents and patent applications have disclosed a method for blowing and depositing copper powder upon a surface of a metal, a method for rubbing and applying copper powder to a surface of a metal, and a method for melting copper powder in stainless steel to obtain copper-stainless steel, etc. Apart from such methods, conventionally known metal copper plating methods may also have been disclosed.

For materials or products where plating methods are applicable, it is easy to coat metallic copper by using plating methods. In this case, materials to be coated inevitably exhibit a copper colored characteristic. However, in the case of materials where plating methods are not easily applied, there have been used a process in which metal copper powder is directly blown to be deposited upon or applied to the surface of a target material or a techniques for applying, to a material of interest, a material in which antibacterial copper powder is dispersed in a resin.

However, even if copper powder is forcibly blown to be deposited or rubbed, it is difficult to uniformly coat a copper film to be fixed on the whole surface of a target material, for example, at a nanometer level. Further, fixed coating tends to be easily delaminated, thus posing a problem associated with preservation and maintenance of antibacterial efficacy.

In addition, even when copper powder is applied after addition of the powder to a coating material or resin, there is also a limit in that antibacterial effects are exerted only at copper powder parts exposed at the surface from the inside of a material structure and antibacterial effects cannot be anticipated at all on copper powder parts that are not exposed at the surface.

Hence, in order to exert antibacterial effects, there has been a need for a stable and sufficient application of copper powder so as to cover the entire surface of a material of interest. However, as well known in the art, there had been no methods capable of covering a material surface with copper only, aside from a copper plating method.

However, even in the case of a material in which the copper plating method can be employed, all of the target materials inevitably exhibit a characteristic copper color, thus limiting the selection and realization of desired product colors. In addition, copper-plated products tend to easily undergo partial discoloration, thus inhibiting the marketability of such products.

Due to the above-mentioned problems, application of the conventional copper coating method has been restricted to materials in which the copper plating method can be used while having no problem even when resulting products are colored with copper color. Therefore, it can be said that there had been little feasibility of various applications to a product such as food containers, ceramics and mineral materials.

### SUMMARY OF THE INVENTION

Therefore, the present invention has been made in view of the above problems, and it is an object of the present invention to provide an antibacterial material exhibiting constant, stable, and potent antibacterial, bactericidal and antifungal effects by formation of uniform, hard and thin film-like copper oxide coatings on entire surfaces of inoxidizable natural materials including mineral materials such as ceramics, glass, stone, tile, pumice and sand, to which a plating method is not easily applicable, or ceramic products and titanium products.

It is another object of the present invention to provide an inexpensive antibacterial material capable of exerting antibacterial effects with use of a very small amount of copper, in exhibiting constant, stable, and potent antibacterial, bactericidal and antifungal effects by formation of uniform, hard and thin film-like copper oxide coatings on entire surfaces of target materials.

It is a further object of the present invention to provide an antibacterial material capable of exhibiting antibacterial effects while undergoing minimal discoloration of materials via use of an ultra thin film of copper oxide, in exhibiting constant, stable, and potent antibacterial, bactericidal and antifungal effects by formation of uniform, hard and thin film-like copper oxide coatings on entire surfaces of target materials.

In accordance with an aspect of the present invention, the above and other objects can be accomplished by the provision of a process for preparing a copper oxide-coated antibacterial material, comprising dissolving less than or equal to 10% by weight of inorganic copper salt powder or copper sulfate hydrate powder in a polar solvent, in relation to the weight of the polar solvent, so as to prepare a copper ion mixed solution, dipping a target material in the mixed solution, and sintering the target material in which copper ions in the mixed solution were deposited onto the surface of the target material, at a predetermined temperature under atmospheric pressure to form a copper oxide coating on the surface of the target material by changing a copper oxide ionic film into copper oxide coating.

In accordance with another aspect of the present invention, there is provided a process for preparing a copper oxide-coated antibacterial material, comprising dissolving less than or equal to 10% by weight of inorganic copper salt powder or copper sulfate hydrate powder in a polar solvent, in relation to the weight of the polar solvent, so as to prepare a copper ion mixed solution, applying the mixed solution to a surface of a target material, and sintering the target material to which the mixed solution was applied, at a predetermined temperature under atmospheric pressure to form a copper oxide coating on the surface of the target material.

Preferably, the step of preparing the mixed solution includes dissolving 0.01% to 10% by weight of inorganic copper salt powder or copper sulfate hydrate powder in the polar solvent.

Preferably, the step of preparing the mixed solution may further include adding a photocatalyst solution to the mixed solution.

The photocatalyst solution may contain titanium dioxide (TiO₂).

The inorganic copper salt compound may be selected from the group consisting of copper nitrate, copper bromide, copper chloride and copper thiocyanate.

The polar solvent may be water or alcohol.

In addition, in accordance with the present invention, the weight % of copper contained in the inorganic copper salt compound is optionally controlled in relation to the polar solvent, such that a color difference value, ΔE, between surface colors of the target material before and after formation of the copper oxide coating is less than or equal to 5.

### BRIEF DESCRIPTION OF THE DRAWINGS

The above and other objects, features and other advantages of the present invention will be more clearly understood from the following detailed description taken in conjunction with the accompanying drawings, in which:

FIG. 1 schematically shows a process flow diagram for preparing a copper oxide-coated antibacterial material in accordance with Embodiment 1 of the present invention; and

FIG. 2 schematically shows a copper oxide-coated antibacterial material prepared by the process for implementing Embodiment 1 of FIG. 1.

### DESCRIPTION OF THE PREFERRED EMBODIMENTS

The preferred embodiments of the present invention will be described in more detail with reference to the accompanying drawings.

### Embodiment 1

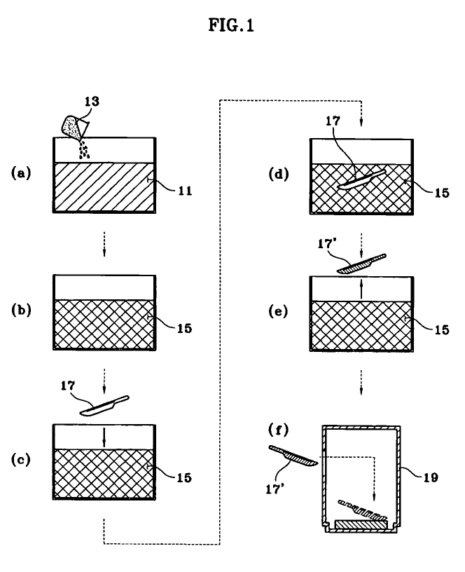

FIG. 1 schematically shows a process flow diagram for preparing an antibacterial material in accordance with this embodiment, for purpose of illustration.

Referring to FIG. 1, in steps (a) and (b), an inorganic copper salt compound 13 is dissolved and mixed in a polar solvent 11 such as water or an alcohol, in an amount of less than or equal to 10% by weight in relation to the weight of the polar solvent 11, thereby preparing a mixed solution 15. The higher the weight % of the inorganic copper salt compound 13 in relation to the weight of the polar solvent 11, the better the resulting antibacterial properties. However, if the content of the inorganic copper salt compound 13 exceeds 10% by weight, this is economically disadvantageous and also does not significantly improve antibacterial properties, compared to when the content of the inorganic copper salt compound 13 is 10% by weight. Therefore, the inorganic copper salt compound 13 is preferably less than or equal to 10% by weight in relation to the polar solvent 11.

In addition, the inorganic copper salt compound 13 that can be used in this embodiment, may be the following inorganic copper salt powder or hydrate powder thereof, for example, copper sulfate hydrate powder, containing more than 10% of a copper ingredient which can be present as copper ions in a solution.

Kinds of inorganic copper salt compound 13 utilized in this embodiment

1) Copper (II) nitrate trihydrate <Cu25%>
Cu(NO₃)₂.3H₂O FW: 241.60

2) Copper (II) bromide <Cu28.5%>
CuBr₂ FW: 223.35

3) Copper (II) chloride dihydrate <Cu37.3%>
CuCl₂.2H₂O FW: 170.48

4) Copper (II) sulfate pentahydrate <Cu25.5%>
CuSO₄.5H₂O FW: 249.69

In steps (c) through (e), for example, the target material 17 such as the ceramic blade of a knife is dipped in and taken out from a mixed solution 15 to obtain the target material 17′ to which a copper ion-containing mixed solution 15 is deposited upon the surface thereof. In this manner, it is possible to prepare a uniform thin film by dipping the target material 17 in a solution. Consequently, the uniform copper oxide coating at the nanometer level can also be obtained in a sintering process that will be described below.

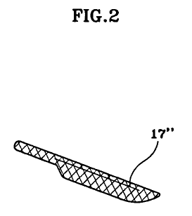

In step (f) of FIG. 1, the target material 17′ is placed into a sintering furnace 19 and sintered at a predetermined temperature. Accordingly, copper ions, present in the mixed solution 15 deposited onto the target material 17′, undergo a thermochemical reaction and thus are fixed to the surface of the target material 17′ in the form of a copper oxide film. As a result, the copper oxide-coated antibacterial material 17″ shown in FIG. 2 is obtained.

That is, the film of the mixed solution 15 deposited onto the surface of the target material 17′ undergoes formation of a copper oxide coating by thermochemical changes through the sintering process in the atmosphere, thereby being strongly and firmly fixed to the surface of the target material 17′. Therefore, the thus formed copper oxide coating becomes free of properties possessed by the original inorganic copper salt compound. Therefore, the copper oxide coating is not dissolved again in water or alcohols and is fixed in the form of a hard coating on the surface of the target material as the inorganic copper oxide coating, thus exerting antibacterial effects.

In addition, when the target material 17 is a ceramic material or metal material, a sintering temperature in the sintering process is preferably within the range of 200 to 900° C. Where the sintering temperature is less than 200° C., formation of a copper oxide coating by copper ions is not sufficiently progressed. In contrast, where the sintering temperature exceeds 900° C., the copper oxide coating formed on the target material 17′ is degraded. Therefore, in both cases that the sintering temperature is outside the above-mentioned range, desired antibacterial effects cannot be anticipated.

The copper oxide coating, fixed on the copper oxide-coated antibacterial material 17″ that is obtained by the processes of this embodiment, has a pencil hardness of more than 9H, as compared to that of 4H exhibited by conventional copper, and therefore is less damaged by scratch or physical impact and is capable of exerting semi-permanent antibacterial effects.

Further, in accordance with the present embodiment, using only a small amount of the inorganic copper salt, in particular, about 0.01% by weight in relation to the polar solvent such as water or alcohol, it is possible to realize superior antibacterial effects at lower costs.

Consequently, in accordance with the preferred embodiment of the present invention, it is possible to produce the copper oxide-coated antibacterial material capable of implementing desired effects of the present invention by use of the inorganic copper salt compound or copper sulfate hydrate in the range of 0.01 to 10% by weight in relation to the polar solvent.

Further, due to the use of a small quantity of copper, the color of copper oxide per se is hardly revealed to the outside, and thus, even when the process of this embodiment is applied to white colored target materials such as ceramics, desired antibacterial properties can be obtained with little change in color unique to the target materials. Hence, the process of this embodiment is advantageously applicable to ceramic products such as artificial teeth, which are susceptibly responsive to discoloration and attach importance to color and tone.

In addition, in accordance with the present embodiment, since the mixed solution in which the inorganic copper salt compound was dissolved in the polar solvent is chemically changed to copper oxide coating and is fixed to the entire surface of the target material by sintering, antibacterial effects can be exerted over the entire surface of the target material.

Additionally, conventional antibacterial technologies utilizing copper involve complicated and troublesome processes. The technologies in accordance with the present embodiment have an advantage in that a target material having excellent antibacterial effects can be easily obtained by simply dipping the target material in the mixed solution, followed by sintering at a given temperature.

### Embodiment 2

This embodiment can be applied to large target materials that are not suitable for dipping in a mixed solution, such as bathtubs. Here, the mixed solution is directly applied to the target material, unlike Embodiment 1, in which the target material is dipped in the mixed solution. Therefore, this embodiment is characterized in that steps (c) through (e), among the steps shown in FIG. 1, are replaced with a step of applying the mixed solution 15 to the target material 17, aside from which the remaining steps and the corresponding effects are the same as those of Embodiment 1.

### Embodiment 3

This embodiment is carried out by using the same procedure as in Embodiments 1 and 2, but is characterized in that it further includes a step of adding a predetermined amount of photocatalyst solution containing, for example, titanium dioxide (TiO₂) to the mixed solution, followed by agitation, prior to dipping a target material in a mixed solution or applying the mixed solution to the target material. Therefore, in accordance with this embodiment, it is possible to obtain antibacterial effects by copper oxide coating, as well as oxidative degradation effects of surface-adhered materials by action of a photocatalyst, thus resulting in prevention of surface contamination of the target material and leading to further enhanced bactericidal effectiveness.

On the other hand, previous embodiments illustrated that the target material is a blade of a knife, but may be applied to any material such as ceramic material, titanium material, glass material, tile, and ceramic product such as tableware, so long as they are target materials that are inoxidizable and withstand a sintering temperature, and may also be applied to bathtubs, sand and stone.

### EXPERIMENTAL EXAMPLE 1

The results in the following Tables 1 and 2 were obtained using test specimens (50×50 mm) of ceramic materials as target materials, and copper (II) nitrate trihydrate as an inorganic copper salt compound. Experimental results show bactericidal effects of various live bacteria for the test specimen of ceramic material (copper oxide-coated antibacterial material) obtained by the procedure of Embodiment 1 using ethanol as the polar solvent.

The term “% value” used in test samples for respective experiments refers to weight % (hereinafter also simply referred to as “solution concentration”) of copper (II) nitrate trihydrate in relation to ethanol. The term “untreated” means that the copper oxide coating was not formed on the ceramic test specimen. The term “non-processed” means that a polyethylene film was used as the test specimen. In addition, the sintering temperature was set at 500° C.

TABLE 1

| | | | Number of Live |
|---|---|---|---|
| | | | Bacteria/Test |
| Test Bacteria | Viable Count | Test Specimen | Specimen |
| E. coli | Immediately after | Non-processed | 1.9 × 10⁵ |
| | inoculation | | |
| | After 24 hours at | Sample 1 | 7.1 × 10⁴ |
| | 35° C. | Sample 2 | <10 |
| | | Sample 3 | <10 |
| | | Sample 4 | <10 |
| | | Sample 5 | <10 |
| | | Sample 6 | <10 |
| | | Non-processed | 2.4 × 10⁷ |

*<10 represents that the number of live bacteria is zero.
Viable counts of E. coli on test specimens
Sample 1: ceramic test specimen (untreated)
Sample 2: ceramic test specimen 1%
Sample 3: ceramic test specimen 0.5
Sample 4: ceramic test specimen 0.1%
Sample 5: ceramic test specimen 0.05%
Sample 6: ceramic test specimen 0.01%

TABLE 2
Viable counts of other bacteria on sample 6

| | | | Number of Live |
|---|---|---|---|
| | | | Bacteria/Test |
| Test Bacteria | Viable Count | Test Specimen | Specimen |
| Staphylococcus | Immediately after | Non-processed | 2.5 × 10⁵ |
| aureus | inoculation | | |
| | After 24 hours at | Non-processed | 1.8 × 10⁵ |
| | 35° C. | Sample 6 | <10 |
| Salmonella | Immediately after | Non-processed | 2.1 × 10⁵ |
| | inoculation | | |
| | After 24 hours at | Non-processed | 7.0 × 10⁵ |
| | 35° C. | Sample 6 | <10 |
| MRSA | Immediately after | Non-processed | 2.3 × 10⁵ |
| | inoculation | | |
| | After 24 hours at | Non-processed | 6.9 × 10⁵ |
| | 35° C. | Sample 6 | <10 |
| Vibrio | Immediately after | Non-processed | 1.6 × 10⁵ |
| parahaemolyticus | inoculation | | |
| | After 24 hours at | Non-processed | 1.5 × 10⁵ |
| | 35° C. | Sample 6 | <10 |
| Legionella | Immediately after | Non-processed | 3.6 × 10⁵ |
| | inoculation | | |
| | After 24 hours at | Non-processed | 5.3 × 10⁵ |
| | 35° C. | Sample 6 | <100 |

As can be seen from Table 1, upon counting and comparing the number of E. coli, immediately after inoculation of E. coli into test specimens and after 24 hours at 35° C., respectively, the non-processed test specimen exhibited more than 100-fold increase in bacterial number, and sample 1) in which the ceramic test specimen was not treated with the copper oxide coating also exhibited little difference in bacterial number, as compared to that counted immediately after inoculation of E. coli. However, samples 2) through 6), in which the ceramic test specimens were coated with the copper oxide coating, exhibited only detection of less than 10 E. coli/test specimen, after 24 hours of E. coli inoculation at 35° C.

As can also be seen from Table 2, when the copper (II) nitrate trihydrate was used in an amount of 0.01% by weight in relation to ethanol, the resulting copper oxide-coated antibacterial material was shown to have superior bactericidal effects on various bacteria, i.e., Staphylococcus aureus, as well as other bacteria such as Salmonella, MRSA, Vibrio parahaemolyticus and Legionella.

### EXPERIMENTAL EXAMPLE 2

It was demonstrated through Experimental Example 1 that the present invention could provide superior antibacterial effects with use of a small amount of an inorganic copper salt compound. However, even though the copper oxide-coated antibacterial material obtained by the present invention has antibacterial effects, it is difficult to commercialize the present antibacterial material as the characteristic color imparted upon products treated with the antibacterial material makes it appear that the product suffers from surface contamination.

Meanwhile, the color of copper oxide is nearly black in color, and thus, when it is applied to artificial teeth made of ceramic material, for example, use of 10% solution concentration causes the artificial teeth to turn black, thus being of no practical use. In addition, in the case of white colored target materials, the higher solution concentration naturally results in greater degree of discoloration. Therefore, this experimental example was carried out to determine an upper limit of the solution concentration that is capable of exerting antibacterial effects with little discoloration before and after processing, in particular, white color type of target materials.

Table 3 below shows experimental results on the color difference between the original target material 17, copper oxide-coated antibacterial material 17″ and non-processed ceramic material, with respect to amount (weight %) of copper nitrate trihydrate, as the inorganic copper salt compound, added to ethanol. The color difference refers to the value representing difference between two different colors, i.e., a sample color and a reference color, and is expressed as ΔE. Color difference of less than 5.0 is the degree to which discoloration is not heavy and thus does not adversely harm the product in an aesthetic sense. A color difference of less than 2.0 refers to a degree in which the presence or absence of discoloration is not perceptible to the naked eye. Meanwhile, the procedure for processing the target material is the same as in Experimental Example 1.

TABLE 3
Reference color = non-processed product/0.00

| | Weight % of Copper Nitrate | Color |
|---|---|---|
| | Trihydrate added (addition | Difference |
| Material to be processed | concentration in ethanol) | Index (ΔE) |
| Ceramics (white zirconium) | (Non-processed product) 0% | 0.00 |
| | 0.01% | 1.61 |
| | 0.05% | 2.80 |
| | 0.1% | 2.98 |
| | 0.5% | 3.72 |
| | 1% | 4.47 |
| | 10% | 20.95 |
| Titanium (black metal) | (Non-processed product) 0% | 0.00 |
| | 0.001% | 0.53 |
| | 0.005% | 1.00 |
| Black Ceramics | (Non-processed product) 0% | 0.00 |
| | 10% | 0.75 |

As can be seen from Table 3, the white ceramic product exhibited a color difference of less than 3 at a solution concentration of 0.01 to 0.10%, and thus, the target material exhibited little discoloration before and after processing, thus being of practical use. In particular, it could be seen that when the solution concentration was 0.01%, the color difference was 1.61, thus a degree in which the presence or absence of discoloration is not discernable by the naked eye. However, when the solution concentration was 10%, the color difference was 20.95, thus representing very heavy discoloration. Hence, for white target materials, the solution concentration is preferably in the range of 0.01 to 0.10%. As such, with the process for preparing the antibacterial material in accordance with the present invention, use of very small quantities of copper can exert antibacterial effects without discoloration of the original target material. Therefore, the present invention is also advantageously applicable to white color type of ceramic products, which are susceptible to discoloration and attach importance to color and tone.

In the case of black colored ceramics, the color difference was only 0.75 even when the solution concentration was 10%, where the thicker copper oxide coating was formed, compared to the white color series of target materials, thus making it possible to obtain excellent effects in magnitude and stability of antibacterial activity. However, as described above, the solution concentration exceeding 10% has no practical use from an economic point of view. Thus, it is preferred to adjust the solution concentration less than 10%.

### EXPERIMENTAL EXAMPLE 3

Table 4 below shows that when the sintering temperature exceeds 900° C., which is an upper limit in the sintering process in accordance with the present invention, the copper oxide coating is thermally degraded, and thus, desired antibacterial effects cannot be obtained. This experimental example used the white colored ceramic specimen with a solution concentration of 1%, and the processing procedure is the same as in Experimental Example 1.

TABLE 4

| | | Color Difference Index |
|---|---|---|
| Test Specimen | Sintering Temperature | (^{Δ}E) |
| White colored ceramic | 500 C. | 5.20 |
| test specimen, 1% | | |
| | 1,000° C. | 1.55 |

As shown in Table 4, the test specimen sintered at 500° C. exhibited a color difference of 5.20 that is normally anticipated. The test specimen sintered at 1000° C. exhibited a color difference of 1.55, thus representing a significant decrease in color difference, as compared to when it was processed at a sintering temperature of 500° C. These results are due to the fact that the copper oxide coating is degraded on the surface of the target material when the sintering temperature is 1000° C. and, thus, show that the desired copper oxide coating cannot be fixed on the target material at sintering temperatures exceeding 900° C.

As apparent from the above description, in accordance with the present invention, there is provided effects of obtaining an antibacterial material exhibiting constant, stable, and potent antibacterial, bactericidal and antifungal effects by formation of uniform, hard and thin film-like copper oxide coatings on entire surfaces of inoxidizable natural materials including mineral materials such as ceramics, stone, tiles, pumice and sand, to which a plating method is not easily applicable, or of ceramic products and titanium products.

Further, in accordance with the present invention, there is also provided effects of obtaining an antibacterial material capable of exhibiting antibacterial effects with little discoloration of the color sense by use of a (very) small amount of an inorganic copper salt compound, in exhibiting constant, stable, and potent antibacterial, bactericidal and antifungal effects by formation of uniform, hard and thin film-like copper oxide coatings on entire surfaces of target materials.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A process for preparing a copper oxide-coated antibacterial material, comprising:
dissolving less than or equal to 10% by weight of inorganic copper salt powder or copper sulfate hydrate powder in a polar solvent, in relation to the weight of the polar solvent, so as to prepare a copper ion mixed solution;
dipping a target material in the mixed solution; and
sintering the target material in which copper ions in the mixed solution were deposited upon the surface of the target material, at a predetermined temperature under atmospheric pressure to form a copper oxide coating on the surface of the target material by change of a copper oxide ionic film into copper oxide coating.

2. The process according to claim 1, wherein the step of preparing the mixed solution includes dissolving 0.01% to 10% by weight of inorganic copper salt powder or copper sulfate hydrate powder into the polar solvent.

3. The process according to claim 1, wherein the step of preparing the mixed solution further includes adding a photocatalyst solution to the mixed solution.

4. The process according to claim 3, wherein the photocatalyst solution contains titanium dioxide (TiO₂).

5. The process according to claim 1, wherein the inorganic copper salt compound is selected from the group consisting of copper nitrate, copper bromide, copper chloride and copper thiocyanate.

6. The process according to claim 1, wherein the polar solvent is water or an alcohol.

7. The process according to claim 1, wherein weight % of copper contained in the inorganic copper salt compound is optionally controlled in relation to the polar solvent, such that a color difference value, ΔE, between surface colors of the target material before and after formation of the copper oxide coating is less than or equal to 5.

8. The process according to claim 1, wherein the sintering temperature is within the range of 200 to 900° C.

9. A process for preparing a copper oxide-coated antibacterial material, comprising:
dissolving less than or equal to 10% by weight of inorganic copper salt powder or copper sulfate hydrate powder in a polar solvent, in relation to the weight of the polar solvent, to prepare a copper ion mixed solution;
applying the mixed solution to a surface of a target material; and
sintering the target material to which the mixed solution was applied, at a predetermined temperature under atmospheric pressure to form a copper oxide coating on the surface of the target material.

10. The process according to claim 9, wherein the step of preparing the mixed solution includes dissolving 0.01% to 10% by weight of inorganic copper salt powder or copper sulfate hydrate powder into the polar solvent.

11. The process according to claim 9, wherein the step of preparing the mixed solution further includes adding a photocatalyst solution to the mixed solution.

12. The process according to claim 11, wherein the photocatalyst solution contains titanium dioxide (TiO₂).

13. The process according to claim 9, wherein the inorganic copper salt compound is selected from the group consisting of copper nitrate, copper bromide, copper chloride and copper thiocyanate.

14. The process according to claim 9, wherein the polar solvent is water or an alcohol.

15. The process according to claim 9, wherein weight % of copper contained in the inorganic copper salt compound is optionally controlled in relation to the polar solvent, such that a color difference value, ΔE, between surface colors of the target material before and after formation of the copper oxide coating is less than or equal to 5.

16. The process according to claim 9, wherein the sintering temperature is within the range of 200 to 900° C.
